(19) United States
(12) Patent Application Publication
(10) Pub. No.: US 2020/0286613 A1
(43) Pub. Date: Sep. 10, 2020

(54) DETECTING TUBE OUTPUT ROLL OFF

(71) Applicant: Hologic, Inc., Marlborough, MA (US)

(72) Inventor: Alan Rego, Woodbury, CT (US)

(73) Assignee: Hologic, Inc., Marlborough, MA (US)

(21) Appl. No.: 16/809,272

(22) Filed: Mar. 4, 2020

Related U.S. Application Data
(60) Provisional application No. 62/813,544, filed on Mar. 4, 2019.

(51) Int. Cl. G16H 30/20; G06N 20/00

(52) U.S. Cl. CPC G16H 30/20; G06N 20/00

(57) ABSTRACT

Examples of the present disclosure describe systems and methods for detecting X-ray tube output roll off (e.g., reduction in X-ray tube radiation output). In aspects, one or more radiation doses may be delivered to an imaging phantom by an X-ray tube as part of a quality control process. The quality control data may be tracked over a period of time and used to update the tube output file corresponding to the X-ray tube. Based on the quality control data and the tube output file updates, the output roll off of the X-ray tube may be detected and tracked at frequent intervals. The frequent tracking of the output roll off may enable the early detection of X-ray tube failure and/or a prediction of an X-ray tube replacement date.

### CROSS-REFERENCE TO RELATED APPLICATIONS

This application claims the benefit of U.S. Provisional Patent Application Ser. No. 62/813,544, filed Mar. 4, 2020, entitled “DETECTING TUBE OUTPUT ROLL OFF,” which application is incorporated herein by reference in its entirety.

### BACKGROUND

X-ray tubes provide an established means for producing and utilizing radiation in the medical, scientific, and inspection fields. As an X-ray tube ages, the radiation output by the X-ray tube is reduced. This reduction in radiation output results in inaccuracies in the radiation dose calculation, and, thus, the radiation dose reported as delivered to patients. Generally, when a reduction in X-ray tube radiation output is detected, the radiation dose calculation is recalibrated or the X-ray tube is replaced. However, the decision to recalibrate or replace an X-ray tube is typically made during a preventative maintenance session of the X-ray device, which occurs infrequently. As a result, inaccurate radiation doses may be reported (recorded with image) for extended periods of time between preventative maintenance sessions.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

Examples of the present disclosure describe systems and methods for detecting X-ray tube output roll off (e.g., reduction in X-ray tube radiation output). In aspects, one or more radiation doses may be delivered to an imaging phantom by an X-ray tube as part of a quality control process. The quality control data may be tracked over a period of time and used to update the tube output file corresponding to the X-ray tube. Based on the quality control data and the tube output file updates, the output roll off of the X-ray tube may be detected and tracked at frequent intervals. The frequent tracking of the output roll off may enable the early detection of X-ray tube failure and/or a prediction of an X-ray tube replacement date.

Aspects of the present disclosure provide a system comprising: at least one processor; and memory coupled to the at least one processor, the memory comprising computer executable instructions that, when executed by the at least one processor, performs a method comprising: receiving imaging data related to a radiation output of an X-ray tube, wherein the imaging data comprises dose values delivered, by the X-ray tube, to an imaging phantom over a period of time; evaluating the data to identify a trend in the dose values; and based on the identified trend, performing an action associated with the X-ray tube.

Aspects of the present disclosure further provide a method comprising: receiving imaging data related to a radiation output of an X-ray tube, wherein the imaging data comprises dose values delivered, by the X-ray tube, to an imaging subject over a period of time; evaluating the data to identify a trend in the dose values; and based on the identified trend, performing an action associated with the X-ray tube.

Aspects of the present disclosure further provide a computer-readable media storing computer executable instructions that when executed cause a computing system to perform a method comprising: receiving imaging data related to a radiation output of an X-ray tube, wherein the imaging data comprises dose values delivered, by the X-ray tube, to an imaging phantom over a period of time; evaluating the data to identify a trend in the dose values, wherein the trend indicates an increase in the dose value over the period of time; and based on the identified trend, determining an amount of output roll off for the X-ray tube.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

Non-limiting and non-exhaustive examples are described with reference to the following figures.

FIG. 1 illustrates an overview of an example system for detecting X-ray tube output roll off, as described herein.

FIG. 2 illustrates an overview of an example input processing system 200 for detecting X-ray tube output roll off, as described herein.

FIG. 3 illustrates an example method for detecting X-ray tube output roll off, as described herein.

FIG. 4 illustrates one example of a suitable operating environment in which one or more of the present embodiments may be implemented.

### DETAILED DESCRIPTION

Medical imaging has become a widely used tool for identifying and diagnosing abnormalities, such as cancers or other conditions, within the human body. Medical imaging processes such as mammography and tomosynthesis are particularly useful tools for imaging breasts to screen for, or diagnose, cancer or other lesions with the breasts. Tomosynthesis systems are mammography systems that allow high resolution breast imaging based on limited angle tomosynthesis. Tomosynthesis, generally, produces a plurality of X-ray images, each of discrete layers or slices of the breast, through the entire thickness thereof. In contrast to typical two-dimensional (2D) mammography systems, a tomosynthesis system acquires a series of x-ray projection images, each projection image obtained at a different angular displacement as the x-ray source moves along a path, such as a circular arc, over the breast. In contrast to conventional computed tomography (CT), tomosynthesis is typically based on projection images obtained at limited angular displacements of the x-ray source around the breast. Tomosynthesis reduces or eliminates the problems caused by tissue overlap and structure noise present in 2D mammography imaging.

Generally, each mammography, tomosynthesis, and CT system includes at least one X-ray tube. The typical life expectancy of an X-ray tube is between 3 to 6 years. However, the life expectancy of an X-ray tube is dependent on several factors, such as the type of imaging procedure performed. For example, tomosynthesis and contrast enhanced mammography stress/degrade an X-ray tube more than conventional 2D mammography. Over the course of an X-ray tube's life, numerous quality control procedures are performed to evaluate the effectiveness or decay of the X-ray tube. For example, radiation dose data may be collected for an X-ray tube during routine imaging phantom testing. By evaluating the historical trend of the imaging phantom testing data, it is possible to detect when the dose being applied to the phantom is reported as drifting up over time. In many cases, the reported dose may have increased due to a decrease in the X-ray tube output. In such cases, when the X-ray tube output has “rolled off” (e.g., decreased), the exposure time is increased when automatic exposure control (AEC) is used. As a result of the increased exposure time, an incorrect dose may be reported by the imaging system. Reliance on the incorrect data results in inaccuracies in the dose calculation for radiation exposure. Although such inaccuracies may be corrected by recalibrating the tube output of the X-ray tube, the recalibration process is performed infrequently by service personnel. As a result, incorrect doses may be reported to patients for extended periods of time between service personnel appointments.

To address such issues with prolonged, inaccurate dose reporting, the present disclosure describe systems and methods for detecting X-ray tube (e.g., X-ray tube) output roll off. In aspects, an X-ray tube may deliver one or more radiation exposures to an imaging phantom during an imaging procedure. An imaging phantom, as used herein, may refer to an object that is scanned or imaged in the medical, scientific, and/or inspection fields to evaluate, analyze, and/or tune the performance of various imaging devices. During an imaging procedures, the radiation dose data for the imaging phantom may be recorded. The recorded radiation dose data may be evaluated over one or more imaging procedures to identify trends. For example, the recorded radiation dose data may indicate that the recorded radiation dose is rising over time. The upward trend in reported radiation dose may indicate that the X-ray tube output has rolled off. Based on the identified trends, one or more remedial actions may be performed. As one example, the X-ray tube output file comprising the static tube output values for the X-ray tube may be updated. As another example, the X-ray tube may be recalibrated or replaced. As yet another example, the amount of remaining X-ray tube life may be calculated, tracked, and/or used to predict a date for an X-ray tube replacement. As still yet another example, an identified trends may be used to track the performance of a particular batch, brand, or model of X-ray tubes. In aspects, although the remedial actions may be performed during routine preventative maintenance sessions (which are often infrequent), the trend identification process may enable issues with an X-ray tube to be identified and addressed between preventative maintenance sessions.

Accordingly, the present disclosure provides a plurality of technical benefits including but not limited to: using imaging phantom dose data to detect and/or model X-ray tube decay; increasing the accuracy of the AEC performance; reporting dose data more accurately and consistently; improving the X-ray tube quality control process; providing remedial actions for detected X-ray tube decay; and reducing the cost associated with the recalibration and/or replacement of image devices (or components thereof).

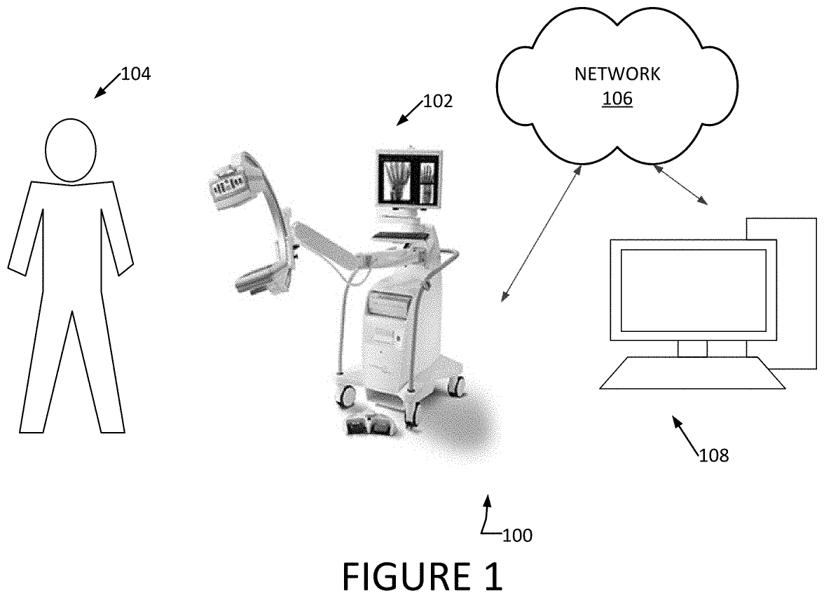

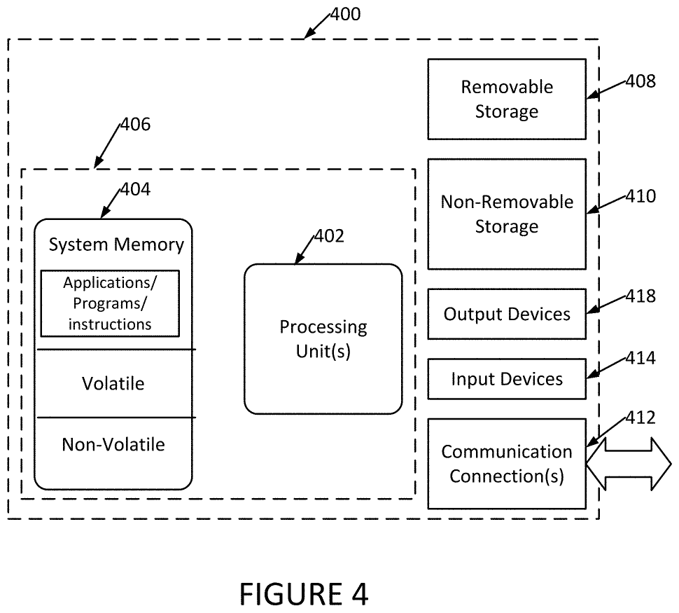

FIG. 1 illustrates an overview of an example system for detecting X-ray tube output roll off as described herein. Example system 100 presented is a combination of interdependent components that interact to form an integrated whole for implementing an enhanced content sharing platform. Components of the system may be hardware components (e.g., used to execute/run operating system (OS)) or software components (e.g., applications, application programming interfaces (APIs), modules, virtual machines, runtime libraries, etc.) implemented on, and/or executed by, hardware components of the system. In one example, example system 100 may provide an environment for software components to run, obey constraints set for operating, and utilize resources or facilities of the system 100. For instance, software may be run on a processing device such as a personal computer (PC), mobile device (e.g., smartphone/phone, tablet, laptop, personal digital assistant (PDA), etc.), and/or any other electronic devices. As an example of a processing device operating environment, refer to the example operating environments depicted in FIG. 4. In other examples, the components of systems disclosed herein may be distributed across multiple devices. For instance, input may be entered on a client device and information may be processed or accessed using other devices in a network, such as one or more server devices.

As one example, the system 100 may comprise imaging device 102, imaging subject 104, network 106, and input processing system 108. One of skill in the art will appreciate that the scale of systems such as system 100 may vary and may include more or fewer components than those described in FIG. 1. For instance, in some examples, imaging device 102 and input processing system 106 may be located in the same device. Alternately, the functionality and components of imaging device 102 and/or input processing system 106 may be distributed across one or more devices.

In aspects, imaging device 102 may be configured to generate a visual representation of the interior of an object, such as imaging subject 104, or the functions of organs and/or tissues thereof. Imaging subject 104 may represent at least a portion of a living being (e.g., a human, an animal, etc.) or an inanimate object (e.g., an imaging phantom or a similar performance analysis object). Imaging device 102 may comprise one or more X-ray tubes (not pictured) and/or corresponding radiation detector mechanisms. As a particular example, imaging device 102 may comprise a gantry assembly. The gantry assembly may be configured as a circular, rotating frame comprising an X-ray tube mounted on one side of the frame and an X-ray detector located on the opposite side of the frame. As the rotating frame rotates the X-ray tube and X-ray detector around the imaging subject, several sectional views of the imaging subject may be generated. The sectional views may then be processed by imaging device 102 to create a two- or three-dimensional visualization of the imaging subject.

In aspects, radiation data may be collected for one or more scans performed by imaging device 102. The radiation data may include, among other things, the dose of radiation (measured in milliRems (mR)) output by an X-ray tube for the duration of an exposure (measured in milliAmpere-second (mAs)). The radiation data may be stored locally by imaging device 102 and/or transmitted via a network, such as network 106, to a separate processing system, such as input processing system 108. Input processing system 108 may be configured to store, process, and/or analyze the radiation data. Based on the analysis of the radiation data, input processing system 108 may identify one or more trends. For example, input processing system 108 may use one or more statistical modeling techniques to determine that the radiation dose provided by an X-ray tube has increased over a period of time. In some aspects, in response to identifying one or more trends in the radiation data, input processing system 108 may cause one or more actions to be performed. For instance, input processing system 108 may calculate an estimated failure date for one or more components of imaging device 102, cause a maintenance appointment to be scheduled, transmit a warning or message of the identified trend to imaging device 102, or generate a status report of the imaging device 102.

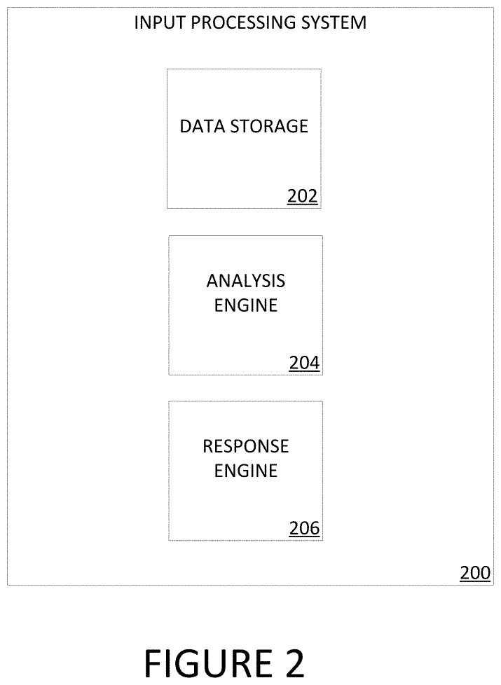

FIG. 2 illustrates an overview of an example input processing system 200 for detecting X-ray tube output roll off, as described herein. The output roll off detection techniques implemented by input processing system 200 may comprise the output roll off techniques and data described in the system of FIG. 1. In some examples, one or more components (or the functionality thereof) of input processing system 200 may be distributed across multiple devices and/or systems. In other examples, a single device (comprising at least a processor and/or memory) may comprise the components of input processing system 200.

With respect to FIG. 2, input processing system 200 may comprise data storage 202, analysis engine 204, and response engine 206. Data storage 202 may be configured to store data related to one or more imaging devices or systems. The data may comprise, for example, date/time information, imaging device and/or component information, procedure information, imaging object data (e.g., patient information, treatment information, etc.), radiation data, dose data, preventative maintenance information (e.g., date of last maintenance, days until next maintenance, previous maintenance report, etc.), or the like.

Analysis engine 204 may be configured to process and/or analyze received data. In aspects, analysis engine 204 may have access to the data stored in data storage 202. Upon accessing the data, analysis engine 204 may analyze the data using one or more analysis rule sets, algorithms or models. A model, as used herein, may refer to a predictive or statistical model that may be used to determine a probability distribution over one or more character sequences, classes, objects, result sets or events, and/or to predict a response value from one or more predictors. A model may be based on, or incorporate, one or more rule sets, machine learning, a neural network, or the like. In aspects, the analysis of the data may identify statistically relevant information, such as treatment responses, data trends, data discrepancies, and the like. As a specific example, the data may indicate that the radiation dose recorded for a particular imaging system has increased over a time period. The data may also indicate whether the increase is linear, exponential, etc. and/or whether any particular events or uses are especially relevant to the increase. Based at least on this data, analysis engine 204 may determine that the output of the X-ray tube of an imaging device has declined (e.g., rolled off). In at least one aspect, analysis engine 204 may output an analysis of the data. The output may be stored in a storage location, such as data storage 202, or transmitted to a separate system or component of input processing system 200.

Response engine 206 may be configured to cause one or more actions to be performed. In aspects, response engine 206 may receive (or otherwise have access to) the analysis data generated/output by analysis engine 204. For example, analysis engine 204 may provide the analysis data to response engine 206 as part of the analysis process. Alternately, response engine 206 may be invoked on demand or as part of a scheduled computing task. In such an example, the analysis data to be evaluated may be selected automatically or manually using a user interface provided by input processing system 200. In aspects, based on the analysis data, response engine 206 may perform (or cause the performance of) one or more actions. For instance, continuing with the above example, in response to an indication that the radiation dose recorded for a particular imaging system has increased over a time period, response engine 206 may automatically generate a warning. The warning may be transmitted to a user and/or an administrative of the imaging device(s) responsible for the data stored in data storage 202. In another example, response engine 206 may use the analysis data to track and evaluate the performance of multiple imaging systems and/or imaging system components. For instance, the analysis data for a single imaging device may cause an analysis to be executed for similar imaging devices, or for imaging devices comprising similar components to the single imaging device

Having described various systems that may be employed by the aspects disclosed herein, this disclosure will now describe one or more methods that may be performed by various aspects of the disclosure. In aspects, method 300 may be executed by an example system, such as system 100 of FIG. 1 or input processing system 200 of FIG. 2. In examples, method 300 may be executed on a device comprising at least one processor configured to store and execute operations, programs or instructions. However, method 300 is not limited to such examples. In other examples, method 300 may be performed on an application or service for detecting X-ray tube output roll off. In at least one example, method 300 may be executed (e.g., computer-implemented operations) by one or more components of a distributed network, such as a web service/distributed network service (e.g. cloud service).

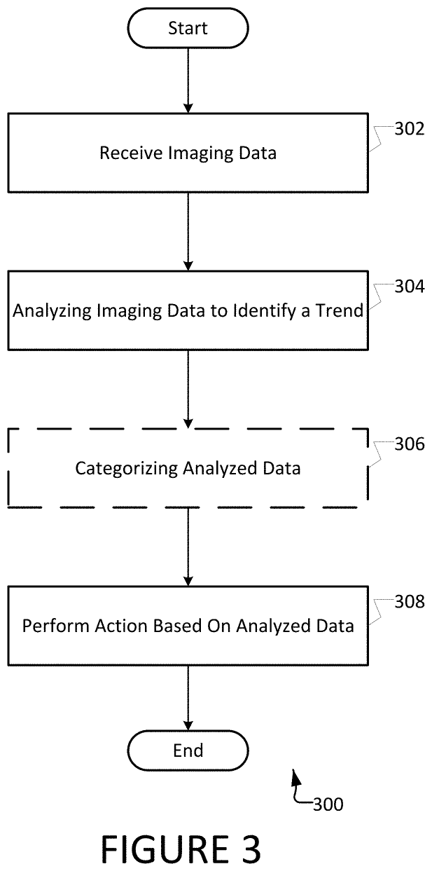

FIG. 3 illustrates an example method 300 for detecting X-ray tube output roll off, as described herein. Example method 300 begins at operation 302, where data is received. In aspects, data relating to one or more imaging devices or systems, such as imaging device 102, may be received. The data may comprise information identifying (or otherwise related to) the imaging subject (e.g., the patient or imaging phantom), the image device or system, image creation date/time, radiation and/or dose data, or the like. For example, the data may include quality control information for an imaging device. The quality control information represent various days over a period of time, and may comprise radiation measurements for an imaging device and an imaging phantom receiving radiation doses from the imaging device. The radiation measurements may be used by the imaging device to determine the dose to be delivered to the imaging subject.

At operation 304, the received data may be analyzed. In aspects, the received data may be provided to an analysis component, such as analysis engine 204. The analysis component may apply one or more rule sets or algorithms to the received data, or the analysis component may provide the received data to one or more analytical models. The rule sets, algorithms, and/or analytical models may be used to identify statistically relevant data trends or patterns, treatment or procedure results, image device use occurrences, etc. For example, the data for a particular imaging device may be provided to an analytical model that implements machine learning (ML). The analytical model may analyze the provided data and determine that the recorded dose delivered by the imaging device has increased over a time period. The analytical model may further determine the rate of increase over the time period. For instance, the analyzed data may indicate that the recorded dose delivered by the imaging device decreased significantly during a first portion of the time period, decreased approximately linearly during a second portion of the time period, and decreased significantly during a third portion of the time period. In some aspects, the analysis by the analysis component may be performed on demand, according to a predetermined schedule (e.g., daily, weekly, etc.), or upon the satisfaction of one or more criteria.

At optional operation 306, the analyzed data may be categorized and/or attributed. In aspects, the analyzed data may be further analyzed by the analysis component. Alternately, the analyzed data may be provided to a separate analysis component for further analysis. The separate analysis component may be, or provide functionality that is similar to, the analysis component discussed above. The further analysis of the analyzed data may comprise categorizing, labeling, or attributing the trends, patterns, and other activity in the analyzed data. For example, the analysis component may be configured to use ML techniques to identify that a trend or pattern is attributable to the decay or malfunction of one or more components of the imaging device, a misconfigured or incorrectly installed component of the imaging device, a software misconfiguration or settings error, etc. As a result, the trend (or data associated therewith) may be categorized as related to component decay (or something similarly indicative of component failure) and labeled accordingly. The categorization and/or labeling may be performed manually or automatically using rule sets, pattern matching fuzzy logic, or similar techniques. For instance, continuing from example above, the analysis component may identify that the increase in the recorded dose delivered by the imaging device is attributable to the decrease in the output of an X-ray tube of the imaging device. As a result, the data corresponding to the identified increase may be labeled as “tube output decay,” and/or categorized as indicative of X-ray tube output roll off. The labels and/or categorizations may be added to the analyzed data and stored in a data store, such as data storage 202.

At operation 308, an action may be performed based on the analyzed data. In aspects, data analyzed using one or more analysis components may be provided to an event generation component, such as response engine 206. The event generation component may evaluate the analyzed data to identify trends or patterns, categorized or labeled data, and other data features. The evaluation may include the use of parsing operations, pattern matching techniques, and/or a set of rules. Based on the evaluation, one or more actions may be performed. For example, a report detailing a data trend and/or the predicted cause of the trend may be generated and sent to the operator of an imaging device. As another example, an order for a replacement part (such as an imaging device tube) for an imaging device may be automatically submitted to a maintenance and/or parts provider. As yet another example, the amount of remaining X-ray tube life may be calculated and used to estimate a replacement date for the X-ray tube. Calculating the remaining tube life may comprise comparing one or more values in the analyzed data to a threshold value. For instance, the most recent dose values in the analyzed data may be compared to a set of threshold values representing various levels of remaining X-ray tube life. When a dose value exceeds a threshold, the corresponding image tube life value (e.g., 50% remaining, 10% remaining, etc.) may be selected and/or assigned to the dose value. As still yet another example, an analysis of a group of imaging devices or imaging device components may be executed. For instance, in response to a determination in the analyzed data that the output of an imaging tube has rolled off, the batch group identifier for the imaging tube may be identified. Using the batch group identifier, a group or batch of imaging tubes associated with (e.g., created at the same time, created using the same process, created by the same manufacturer, etc.) the imaging tube may be identified. The identified group or batch of imaging tubes may then be analyzed to determine whether similar trends in imaging tube output roll off are detected. In aspects, the actions generated by the event generation component enable the early detection and mitigation of issues with the imaging device(s) and components thereof.

FIG. 4 illustrates an exemplary suitable operating environment for detecting X-ray tube output roll off described in FIG. 1. In its most basic configuration, operating environment 400 typically includes at least one processing unit 402 and memory 404. Depending on the exact configuration and type of computing device, memory 404 (storing, instructions to perform the X-ray tube roll off detection techniques disclosed herein) may be volatile (such as RAM), nonvolatile (such as ROM, flash memory, etc.), or some combination of the two. This most basic configuration is illustrated in FIG. 4 by dashed line 406. Further, environment 400 may also include storage devices (removable, 408, and/or non-removable, 410) including, but not limited to, magnetic or optical disks or tape. Similarly, environment 400 may also have input device(s) 414 such as keyboard, mouse, pen, voice input, etc. and/or output device(s) 416 such as a display, speakers, printer, etc. Also included in the environment may be one or more communication connections 412, such as LAN, WAN, point to point, etc. In embodiments, the connections may be operable to facility point-to-point communications, connection-oriented communications, connectionless communications, etc.

Operating environment 400 typically includes at least some form of computer readable media. Computer readable media can be any available media that can be accessed by processing unit 402 or other devices comprising the operating environment. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory medium which can be used to store the desired information. Computer storage media does not include communication media.

Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, microwave, and other wireless media. Combinations of the any of the above should also be included within the scope of computer readable media.

The operating environment 400 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet.

The embodiments described herein may be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices may be employed to perform the functionality disclosed herein without departing from the scope of the disclosure.

This disclosure describes some embodiments of the present technology with reference to the accompanying drawings, in which only some of the possible embodiments were shown. Other aspects may, however, be embodied in many different forms and should not be construed as limited to the embodiments set forth herein. Rather, these embodiments were provided so that this disclosure was thorough and complete and fully conveyed the scope of the possible embodiments to those skilled in the art.

Although specific embodiments are described herein, the scope of the technology is not limited to those specific embodiments. One skilled in the art will recognize other embodiments or improvements that are within the scope and spirit of the present technology. Therefore, the specific structure, acts, or media are disclosed only as illustrative embodiments. The scope of the technology is defined by the following claims and any equivalents therein.

What is claimed is:

1. A system comprising:
at least one processor; and
memory coupled to the at least one processor, the memory comprising computer executable instructions that, when executed by the at least one processor, performs a method comprising:
receiving imaging data related to a radiation output of an X-ray tube, wherein the imaging data comprises dose values delivered, by the X-ray tube, to an imaging phantom over a period of time;
evaluating the data to identify a trend in the dose values; and
based on the identified trend, generating an action associated with the X-ray tube.

2. The system of claim 1, wherein the imaging data corresponds to quality control data for the X-ray tube.

3. The system of claim 1, wherein the dose values are stored in a file over the period of time, the file storing a value being used to determine a dose to be delivered to the imaging phantom.

4. The system of claim 1, wherein evaluating the data comprises providing the data to an analytical model for identifying statistically relevant patterns in the imaging data.

5. The system of claim 1, wherein evaluating the data comprises applying to the data at least one of a rule set or a statistical model.

6. The system of claim 1, wherein the trend is indicative of a decrease in the radiation output of the X-ray tube.

7. The system of claim 1, the method further comprising:
categorizing the identified trend; and
labeling the trend based on the categorization.

8. The system of claim 1, the method further comprising:
automatically attributing a cause for the identified trend.

9. The system of claim 1, wherein the action is at least one of: generating a report, generating a notification, or scheduling a maintenance appointment for the X-ray tube.

10. A method comprising:
receiving imaging data related to a radiation output of an X-ray tube, wherein the imaging data comprises dose values delivered, by the X-ray tube, to an imaging subject over a period of time;
evaluating the data to identify a trend in the dose values; and
based on the identified trend, generating an action associated with the X-ray tube.

11. The system of claim 10, wherein the imaging data further comprises date/time information and identification information for an imaging device comprising the X-ray tube.

12. The system of claim 10, wherein the imaging subject is at least one of an imaging phantom and a patient.

13. The system of claim 10, wherein evaluating the data comprise applying one or more machine learning (ML) techniques to the data.

14. The system according to claim 10 or 13, wherein the ML techniques are used to determine that the trend is at least one of linear or exponential.

15. The system of claim 10, wherein evaluating the data comprises determining a cause for the trend.

16. The system of claim 10, wherein the action is generated and performed automatically in response to determining the cause for the trend.

17. The system of claim 10, wherein the action is associated with at least one of recalibrating the X-ray tube and replacing the X-ray tube.

18. A computer-readable media storing computer executable instructions that when executed cause a computing system to perform a method comprising:
receiving imaging data related to a radiation output of an X-ray tube, wherein the imaging data comprises dose values delivered, by the X-ray tube, to an imaging phantom over a period of time;
evaluating the data to identify a trend in the dose values, wherein the trend indicates an increase in the dose value over the period of time; and
based on the identified trend, determining an amount of output roll off for the X-ray tube.

19. The computer-readable media of claim 18, wherein determining an amount of output roll off comprises comparing one or more of the dose values to a threshold value.

20. The computer-readable media of claim 18, wherein the threshold value indicates an amount remaining life for the X-ray tube.
